(19) United States
(12) Patent Application Publication
(10) Pub. No.: US 2025/0025208 A1
(43) Pub. Date: Jan. 23, 2025

(54) INSTRUMENT SEAL FOR SURGICAL ACCESS ASSEMBLY

(71) Applicant: Covidien LP, Mansfield, MA (US)

(72) Inventors: Roy J. Pilletere, Middletown, CT (US); Garrett P. Ebersole, Hamden, CT (US); Eric Brown, Madison, CT (US); Matthew A. Dinino, Newington, CT (US); Jacob C. Baril, Norwalk, CT (US); Richard C. Hart, Saint Augustine, FL (US); Justin Thomas, New Haven, CT (US); Nicolette R. LaPierre, Windsor Locks, CT (US)

(73) Assignee: Covidien LP, Mansfield, MA (US)

(21) Appl. No.: 18/904,941

(22) Filed: Oct. 2, 2024

Related U.S. Application Data
(63) Continuation of application No. 18/142,650, filed on May 3, 2023, now Pat. No. 12,127,763.
Application No. 18/142,650 is a continuation of application No. 16/823,503, filed on Mar. 19, 2020, now Pat. No. 11,642,153.

(51) Int. Cl. A61B 17/34

(52) U.S. Cl. CPC A61B 17/3462; A61B 17/3423; A61B 17/3498; A61B 2017/3425

(57) ABSTRACT

A surgical access assembly includes a housing, a tubular member, and a valve assembly. The tubular member extends from the housing. The valve assembly is positioned in the housing and includes a centering mechanism, a guard assembly disposed on a first side of the centering mechanism, and an instrument seal disposed on a second side of the centering mechanism. The instrument seal including petals that are arrange in an overlapping arrangement.

### CROSS-REFERENCE TO RELATED APPLICATION

This application is a continuation of U.S. patent application Ser. No. 18/142,650, filed May 3, 2023, which is a continuation of U.S. patent application Ser. No. 16/823,503, filed on Mar. 19, 2020, now U.S. Pat. No. 11,642,153, the entire disclosures of which are incorporated herein by reference in their entireties.

### FIELD

The present disclosure relates generally to access assemblies including seals for minimally invasive surgery. More particularly, the present disclosure relates to instrument seals for surgical access assemblies.

### BACKGROUND

In order to facilitate minimally invasive surgery, a working space must be created at a surgical site. An insufflation fluid, typically CO₂, is introduced into the abdomen of the patient to create an inflated state called pneumoperitoneum. Surgical access assemblies are utilized to allow the introduction of surgical instrumentation and endoscopes (or other visualization tools). These surgical access assemblies maintain the pressure for the pneumoperitoneum, as they have one or more seals that adapt to the surgical instrumentation. Typically, a “zero-seal” in the surgical access assembly seals the surgical access assembly in the absence of a surgical instrument in the surgical access assembly, and an instrument seal seals around a surgical instrument that has been inserted through the surgical access assembly.

The breadth of surgical instrumentation on the market today requires a robust seal capable adjusting to multiple sizes and withstanding multiple insertions and withdrawals of surgical instrumentation. Some of the surgical instrumentation can include sharp edges that can tear or otherwise damage seals. Therefore, it would be beneficial to have an access assembly with improved seal durability.

### SUMMARY

In an embodiment, a surgical access assembly includes a housing, a tubular member extending from the housing, and a valve assembly disposed in the housing. The valve assembly includes a centering mechanism, a guard assembly, and an instrument seal. The centering mechanism has a central opening. The guard assembly has a central orifice that is alignable with the central opening of the centering mechanism. The guard assembly is disposed on a first side of the centering mechanism. The instrument seal includes a central hole alignable with the central opening of the centering mechanism and is disposed on a second side of the centering mechanism opposite the first side of the centering mechanism and proximate the tubular member. The instrument seal includes petals that are arranged such that a portion of one petal covers a portion of a first adjacent petal and is covered by a portion of a second adjacent petal.

The surgical access assembly may also include a retainer having first and second rings. The first ring may be disposed on the first side of the centering mechanism and the second side may be disposed on the second side of the centering mechanism. The retainer may sandwich the centering mechanism between the guard assembly and the instrument seal. The first ring may include pins extending therefrom and the second ring may include openings for receiving the pins therein.

The central opening of the centering mechanism may be circumscribed by a lip with pores extending therethrough, the guard assembly may include a ring with bores extending therethrough, and the instrument seal may include holes extending therethrough. The pins of the first ring may extend through the bores of the guard assembly, the pores of the centering mechanism, and the holes of the instrument seal to maintain the guard assembly, the centering mechanism, and the instrument seal in an aligned relationship. The pins of the first ring may be received in the openings of the second ring.

The instrument seal may have a frame that defines the central hole. The petals may be flexibly coupled to the frame. The petals may be coupled to the frame with living hinges.

In embodiments, a surgical access assembly has a housing, a tubular member extending from the housing, and a valve assembly disposed in the housing. The valve assembly includes a guard assembly with a central orifice and an instrument seal having a central hole aligned with the central orifice of the guard assembly. The instrument seal includes a frame with petals that are flexibly coupled to the frame. The instrument seal has an unfolded configuration defined by the petals extending away from a center of the frame and a folded configuration defined by the petals folded towards the central hole of the instrument seal such that each petal at least partially overlaps an adjacent petal such that the petals interlock.

The folded configuration of the instrument seal may define a diameter of the central hole that is configured to seal against a surgical instrument.

The petals may be flexibly coupled to the frame with living hinges.

The folded configuration of the instrument seal may allow the petals to flex relative to the frame while the frame may remain axially stationary relative to the housing.

The valve assembly may include a centering mechanism with a central opening. The guard assembly may be disposed on a first side of the centering mechanism and the instrument seal may be disposed on a second side of the centering mechanism that is opposite the first side.

The valve assembly may also include a retainer with first and second rings. The first ring may be disposed on the first side of the centering mechanism and the second ring may be disposed on the second side of the centering mechanism. The retainer may sandwich the centering mechanism between the guard assembly and the instrument seal.

The first ring of the retainer may include pins and the second ring of the retainer may include openings for receiving the pins. The pins of the first ring may be insertable through bores of the guard assembly, pores of the centering mechanism, and holes of the instrument seal to maintain the guard assembly, the centering mechanism, and the instrument seal in an aligned relationship.

In another embodiment, a surgical access assembly has a housing, a tubular member extending from the housing, and a valve assembly disposed in the housing. The valve assembly includes a centering mechanism with a central opening and an instrument seal in an abutting relationship with the centering mechanism. The instrument seal has a frame and petals. A first end of each petal is flexibly coupled to an outer surface of the frame and a second end of each petal is repositionable between a first position where the second end is outside a perimeter of the frame and a second position where the second end is inside the perimeter of the frame. Each petal partially overlaps an adjacent petal such that the petals interlock.

The instrument seal may have a central hole defined by the second position of the petals. The central hole may define a diameter configured to seal against a surgical instrument. The central hole of the instrument seal may be alignable with the central opening of the centering mechanism.

The valve assembly may also include first and second rings. The first ring may be disposed adjacent the centering mechanism and the second ring may be disposed adjacent the instrument seal. The centering mechanism and the instrument seal may be sandwiched between the first and second rings.

The first ring may have pins extending therefrom and the second ring may have openings for receiving the pins therein.

The valve assembly may also include a guard assembly with a central orifice. The guard assembly may be disposed between the first ring and the centering mechanism.

In a further embodiment, a surgical access assembly includes a housing, a tubular member extending from the housing, and an instrument seal disposed in the housing. The instrument seal has a frame with a plurality of frame arms. Each frame arm of the plurality of frame arms is flexibly coupled to at least one other frame arm of the plurality of frame arms. The instrument seal also includes a plurality of petals corresponding to the plurality of frame arms. The plurality of petals is arranged such that a portion of a first petal of the plurality of petals covers a portion of a first adjacent petal of the plurality of petals and is covered by a portion of a second adjacent petal of the plurality of petals. Each petal of the plurality of petals is flexibly coupled to the corresponding frame arm of the plurality of frame arms. The instrument seal further includes a plurality of fins flexibly coupled to the plurality of frame arms. Each fin of the plurality of fins is biased away from the corresponding frame arm of the plurality of frame arms. The plurality of fins is configured to engage an inner surface of the housing.

The plurality of fins may be configured to urge the instrument seal towards a center of the housing.

Movement of the instrument seal relative to a central longitudinal axis of the housing may compress one or more of the fins of the plurality of fins.

The instrument seal may further include a central hole aligned with a central longitudinal axis of the housing. The central hole may be configured to seal against a surgical instrument.

The surgical access assembly may further include a guard assembly with a central orifice. The guard assembly may be disposed on a first side of the instrument seal.

The surgical access assembly may further include a retainer. The retainer may have first and second discs sandwiching the guard assembly and the instrument seal therebetween.

The instrument seal may include orifices extending through each petal of the plurality of petals and the guard assembly may include bores extending therethrough.

The first disc may include pins and the second disc may include openings for receiving the pins. The pins may be insertable through the bores and the orifices to maintain the guard assembly and the instrument seal in an aligned relationship.

Each petal of the plurality of petals may be adapted to flex relative to the frame while the frame remains axially stationary relative to the housing.

### BRIEF DESCRIPTION OF DRAWINGS

Embodiments of an instrument seal are disclosed herein with reference to the drawings, wherein:

FIG. 1 is a perspective view of a surgical access assembly according to an embodiment of the present disclosure;

FIG. 2 is a cross-sectional view of the surgical access assembly of FIG. 1 taken along section line 2-2 of FIG. 1;

FIG. 3 is a top perspective view of an instrument seal in an unfolded configuration according to an embodiment of the present disclosure;

FIG. 4 is a top plan view of the instrument seal of FIG. 3;

FIG. 5 is a side elevational view of the instrument seal of FIG. 4;

FIG. 6 is a top perspective view of the instrument seal of FIG. 4 in a partially folded configuration;

FIG. 7 is a top perspective view of the instrument seal of FIG. 4 in a fully folded configuration illustrating the folding sequence of the petals;

FIG. 8 is a bottom perspective view of a valve assembly according to an embodiment of the present disclosure;

FIG. 9 is an exploded view, with parts separated, of the valve assembly of FIG. 8 including a centering mechanism, a guard assembly, a retainer, and the instrument seal of FIG. 3;

FIG. 10. is a top perspective view of an instrument seal according to an alternate embodiment of the present disclosure;

FIG. 11 is a top perspective view of the instrument seal of FIG. 10 in an unfolded configuration;

FIG. 12 is a top plan view of the instrument seal of FIG. 11 with a link severed;

FIG. 13 is a top plan view of the instrument seal of FIG. 12 in a partially folded configuration illustrating the folding sequence of the petals;

FIG. 14 is a top plan view of the instrument seal of FIG. 12 in a fully folded configuration illustrating the folding sequence of the petals; and

FIG. 15 is a top cross-sectional view of an instrument housing of the surgical access assembly taken along section line 15-15 of FIG. 1 showing the placement of the instrument seal of FIG. 10 disposed therein.

### DETAILED DESCRIPTION

Embodiments of the presently disclosed instrument seal for a surgical access assembly will now be described in detail with reference to the drawings wherein like numerals designate identical or corresponding elements in each of the several views. As is common in the art, the term “proximal” refers to that part or component closer to the user or operator, i.e. surgeon or physician, while the term “distal” refers to that part or component farther away from the user.

Surgical access assemblies are employed during minimally invasive surgery, e.g., laparoscopic surgery, and provide for the sealed access of surgical instruments into an insufflated body cavity, such as the abdominal cavity. The surgical access assemblies of the present disclosure include an instrument valve housing mounted on a cannula tube, and include an obturator (not shown) inserted through the instrument valve housing and cannula tube. The obturator can have a blunt distal end, or a bladed or non-bladed penetrating distal end and can be used to incise the abdominal wall so that the surgical access assembly can be introduced into the abdomen. The handle of the obturator can engage or selectively lock into the instrument valve housing of the surgical access assembly.

Surgical access assemblies with a trocar obturator are employed to tunnel through an anatomical structure, e.g., the abdominal wall, either by making a new passage through the structure or by passing through an existing opening through the anatomical structure. Once the surgical access assembly with the trocar has tunneled through the anatomical structure, the trocar obturator is removed, leaving the surgical access assembly in place. The instrument valve housing of the surgical access assembly includes valves that prevent the escape of insufflation fluid from the body cavity, while also allowing surgical instruments to be inserted into the cavity and minimizing the escape of insufflation fluid.

In various embodiments, a bladeless optical trocar obturator may be provided that permits separation of tissue planes in a surgical procedure and visualization of body tissue fibers as they are being separated, thereby permitting a controlled traversal across a body wall. In other embodiments, the trocar obturator may be bladeless without being optical, e.g., without providing contemporaneous visualization thereof through the distal tip of the obturator. The bladeless obturator may be provided for the blunt dissection of the abdominal lining during a surgical procedure.

Various trocar obturators suitable for use with the surgical access assemblies of the present disclosure are known and include, for example, bladed, bladeless, blunt, optical, and non-optical. For a detailed description of the structure and function of exemplary trocar assemblies, including exemplar trocar obturators and exemplar cannulas, please refer to PCT Publication No. WO 2016/186905 (“the '905 publication”), the content of which is hereby incorporated by reference herein in its entirety.

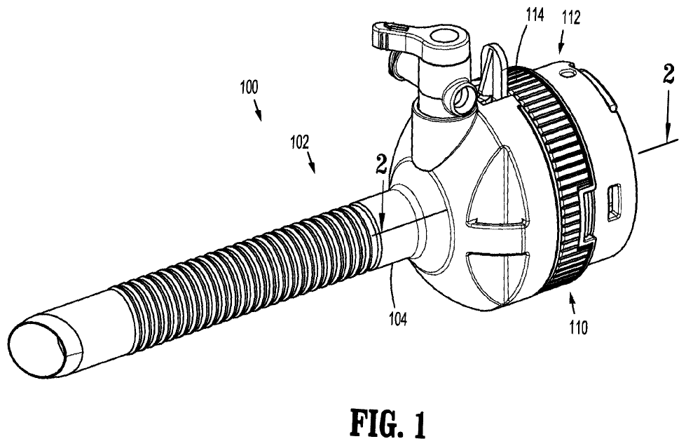

With initial reference now to FIG. 1, a surgical access assembly according to aspects of the present disclosure is shown generally as cannula assembly 100. The cannula assembly 100 includes a cannula 102 and an instrument valve housing 110 secured to the cannula 102. For a detailed description of an exemplary cannula assembly, please refer to the '905 publication.

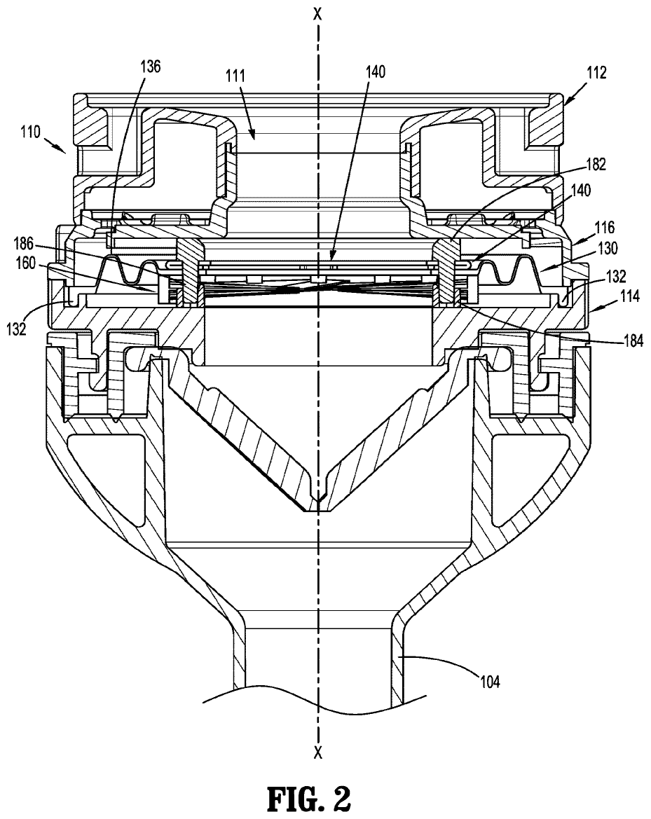

With additional reference to FIG. 2, the instrument valve housing 110 of the cannula assembly 100 includes an upper housing section 112, a lower housing section 114, and an inner housing section 116. The upper, lower, and inner housing sections 112, 114, 116 are configured to support a valve assembly 120 on a proximal end of the cannula 102. More particularly, the inner housing section 116 is secured between the upper and lower housing sections 112, 114, and the valve assembly 120 is received between the inner and lower housing sections 116, 114. The upper and lower housing sections 112, 114 of the instrument valve housing 110 may be selectively attachable to, and detachable from, the inner housing section 116. The lower housing section 114 may be releasably or permanently attached to a cannula tube 104 of the cannula assembly 102. In embodiments, either or both of the upper and lower housing sections 112, 114 of the instrument valve housing 110 may include knurls, indentations, tabs, or be otherwise configured to facilitate engagement by a clinician.

The cannula assembly 100 may also include features for the stabilization of the surgical access assembly. For example, the distal end of the cannula tube 104 can carry a balloon anchor or another expandable member that engages the abdomen from the interior side. For example, see U.S. Pat. No. 7,300,448, the entire disclosure of which is hereby incorporated by reference herein. A feature on the opposite side of the abdominal wall can be used to further stabilize the surgical access assembly, such as adhesive tabs or adjustable foam collars.

The upper, lower, and inner housing sections 112, 114, 116 of the instrument valve housing 110 define a longitudinal passage 111 for receipt of a surgical instrument (not shown). The valve assembly 120 is supported within the instrument valve housing 110 to provide sealed passage of the surgical instrument through the cannula assembly 100.

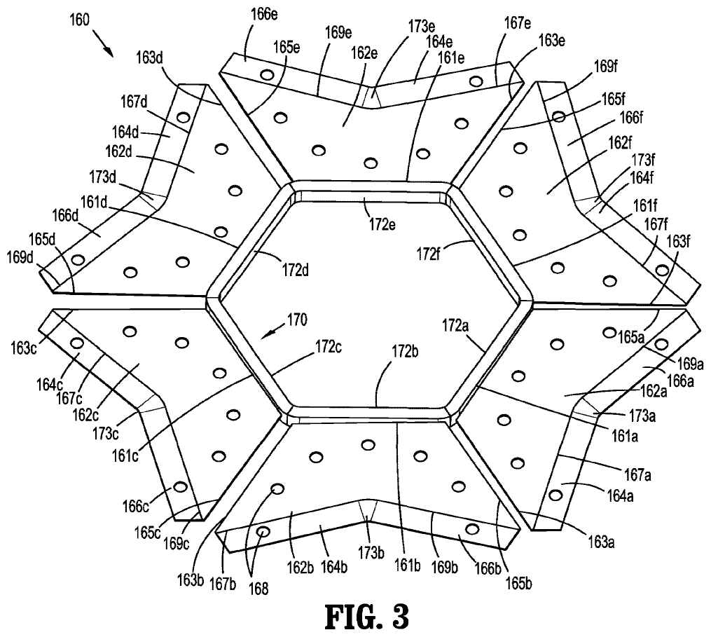

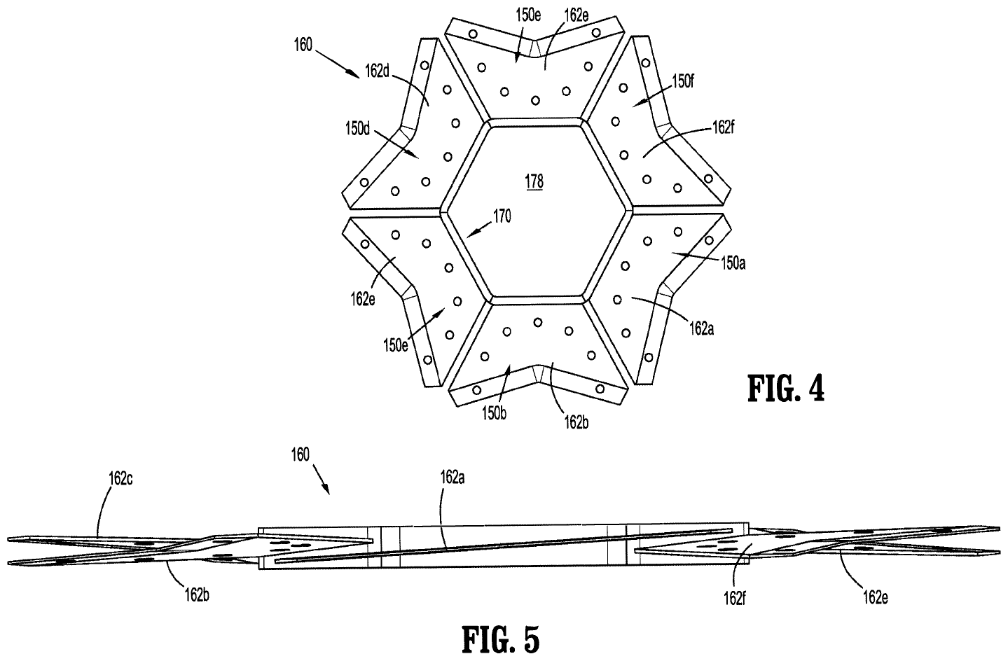

Referring now to FIGS. 3-5, an instrument seal 160, according to an embodiment of the present disclosure, is illustrated. The instrument seal 160, as illustrated, includes a hexagonal frame 170 that may be integrally formed (i.e., monolithic or unitary) or may be formed from six discrete segments that are joined together to form the frame 170. The segments may be joined to each other by welding, adhesives, mechanical joints, or other techniques as known in the art. The sides or segments 172a-f of the frame 170 form a boundary that defines a passage 178 having a center through the instrument seal 160. The center of the passage 178 is coaxial with a central hole 176 of the instrument seal 160. A corresponding number of petals 162 are attached to the frame 170. Although depicted with six petals 162a-f coupled to a hexagonal frame 170, the instrument seal may include a frame with more sides or discrete segments and a corresponding number of petals (e.g., 8). Alternatively, the presently disclosed instrument seal may include a frame with fewer sides or discrete segments and a corresponding number of petals (e.g., 4). The frame 170 and the petals 162a-f may be fabricated from a polyisoprene, a liquid silicone rubber, or another suitable polymeric material. The instrument seal 160 may be molded, stamped, or formed in any other suitable manner. Each petal 162a-f is flexibly coupled to a side 172a-f of the frame 170 via a living hinge 174a-f. Further, as shown in FIGS. 3 and 5, each petal 162a-f is attached to the corresponding side 172a-f of the frame 170 via the living hinge 174a-f such that each petal 162a-f and the corresponding living hinge 174a-f define an acute angle with respect to either a top or bottom surface of the side or segment of the frame 170. The acute angle may be in the range of about 3° to about 10°. By angling each petal 162a-f relative to the top or bottom surface of the frame 170, interweaving the petals 162a-f of the instrument seal 160 is easier than if each petal 162a-f was parallel with the top or bottom surface of the frame 170.

Each petal 162a-f is a five sided main panel 150a-f with holes 168 extending therethrough. Although shown with five sides, each main panel 150a-f may have more or less than five sides. A first or connection side 161a-f is coupled to a side or segment 172a-f of the frame 170 with the corresponding living hinge 174a-f. This arrangement allows the petal 162a-f to be transitioned from an unfolded configuration (FIG. 3) to a folded configuration (FIG. 7). Each living hinge 174a-f may be formed from the same material as the frame 170 and the petals 162a-f or may be formed from another suitable polymeric material. In the unfolded configuration, each petal 162a-f extends away from an outer surface of the frame 170 outside a perimeter defined by the frame 170. In the folded configuration, each petal 162a-f is bounded by the frame 170 and is within the perimeter defined by the frame 170. Each main panel 150a-f has angled second and third sides 163a-f, 165a-f that extend from the connection side 161a-f in a divergent manner. Fourth and fifth sides 167a-f, 169a-f of main panels 150a-f interconnect the angled second and third sides 163a-f, 165a-f. The fourth and fifth sides 167a-f, 169a-f of the main panels 150a-f of each petal 162a-f have equal lengths and are angled towards the corresponding connection side 161a-f such that they meet a point that would bisect the connection side 161a-f. Additionally, the fourth and fifth sides 167a-f, 169a-f are oriented such that they define an angle between 120° and 160°. First and second extenders 164a-f, 166a-f are attached to the fourth and fifth sides 167a-f, 169a-f. Each extender 164a-f, 166a-f includes a hole 168 extending therethrough. The first and second extenders 164a-f, 166a-f have equal lengths and meet at wedges 173a-f that also is located at a point that would bisect the connection side 161a-f. The extenders 164a-f, 166a-f and the main panels 150a-f of each petal 162a-f bend at a midpoint between the second and third sides 163a-f, 165a-f of each petal 162a-f such that, when viewed from the end (i.e., from the extenders towards the connection side) (see FIG. 5), the petal 162a-f has a slight curvature of about 3° to about 10°. The combination of the petals' 162a-f curvature, the angled relationship between each petal 162a-f and the side or segment 172a-f of the frame 170, and the material of construction, facilitates folding the petals 162a-f in an interlocking pattern when transitioning the instrument seal 160 from the unfolded configuration to the folded configuration.

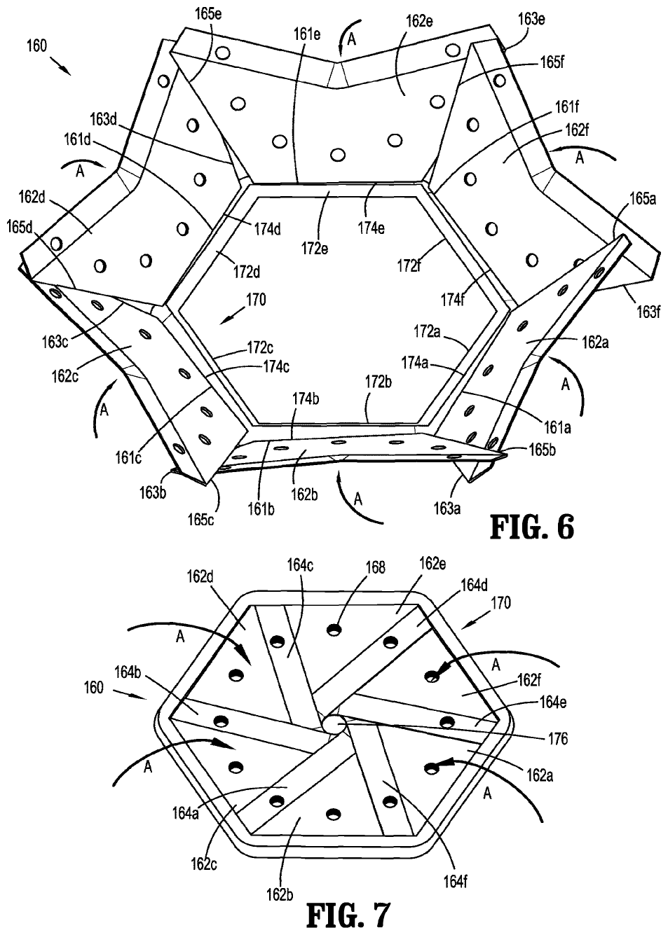

With reference now to FIGS. 3, 6, and 7, transitioning the instrument seal 160 from the unfolded configuration (FIG. 3) to the folded configuration (FIG. 7) includes folding the petals 162a-f sequentially such that they interlock by having each petal 162a-f partially overlap an adjacent petal 162a-f. Initially, as seen in FIG. 3, the instrument seal 160 is in the unfolded configuration with the extenders 164a-f, 166a-f of the petals 162a-f facing away from the frame 170. Each petal 162a-f is folded along a line defined by the associated living hinge 174a-f which defines an angle between the connection side 161a-f of the respective petal 162a-f and the corresponding side or segment 172a-f of the frame 170. Thus, the intersection of the connection side 161a-f and the corresponding third side 165a-f of each petal 162a-f is closer to the side or segment 172a-f of the frame 170 than the intersection of the connection side 161a-f and the second side 163a-f of each petal 162a-f is to the side or segment 172a-f of the frame 170. The petals 162a-f are folded sequentially in the direction of arrows “A” such that all of the petals 162a-f are in a near vertical orientation (FIG. 6). In this arrangement, the second side 163a-f of one petal 162a-f partially overlaps the third side 165a-f of the adjacent petal 162a-f. In particular, as illustrated in FIG. 6, the third side 165a of the first petal 162a partially overlaps the second side 163f of the sixth petal 162f, the third side 165b of the second petal 162b partially overlaps the second side 163a of the first petal 162a, the third side 165 of the third petal 162c partially overlaps the second side of the second petal 162b, the third side 165d of the fourth petal 162d partially overlaps the second side 163c of the third petal 162c, the third side 165e of the fifth petal 162e partially overlaps the second side 163d of the fourth petal 162d, the third side 165f of the sixth petal 162f partially overlaps the second side 163e of the fifth petal 162c, and the third side 165a of the first petal 162a partially overlaps the second side 163f of the sixth petal 162f. This defines a partially folded configuration of the instrument seal 160. Subsequently, the user continues to fold the petals 162a-f towards a center of the frame 170 in the direction of arrows “A” while maintaining the overlapping arrangement between the second and third sides 163a-f, 165a-f of the petals 162a-f. Once all the petals 162a-f are folded such that they are substantially flush with a top surface of the frame 170, the overlapping arrangement of the second and third sides 163a-f, 165a-f of the petals 162a-f maintains the petals 162a-f in contact with one another thereby maintaining the instrument seal 160 in the folded configuration. Further, once all the petals 162a-f are folded over, the holes 168 of the petals 162a-f are aligned thereby allowing pins 186 of a retainer 180 to pass therethrough as will be discussed in detail hereinbelow. As seen in FIG. 7, the folded configuration of the instrument seal 160 defines a central hole 176 for slidably receiving a surgical instrument therethrough. The central hole 176 may have a diameter between 0.025 inches to 0.100 inches (i.e., 0.0635 cm to 0.254 cm).

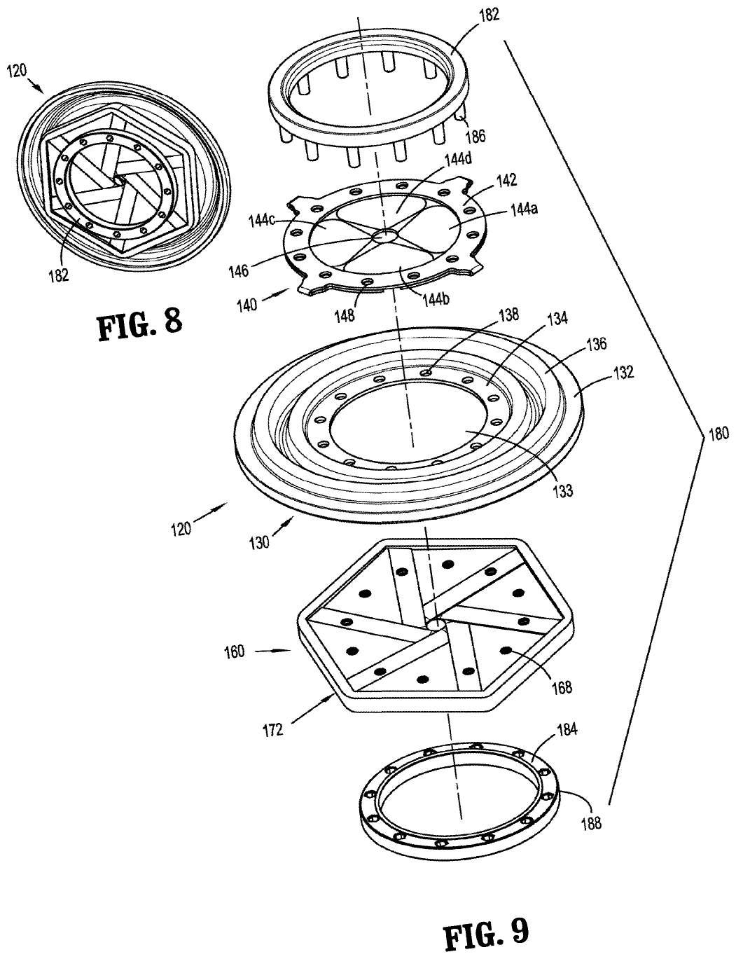

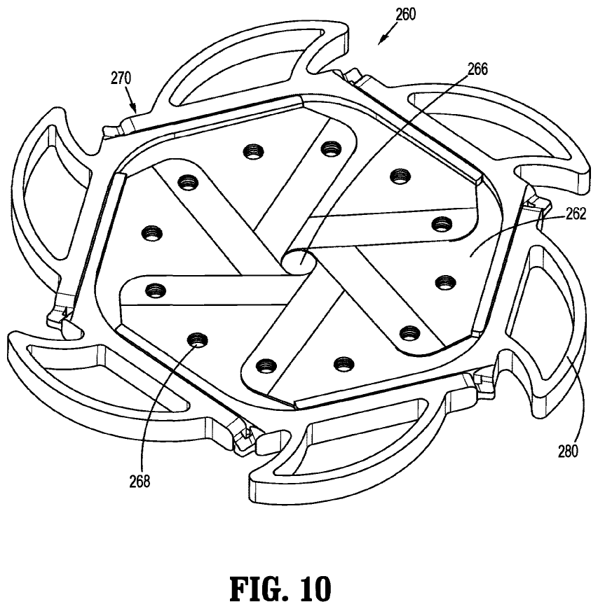

With reference now to FIGS. 2, 8, and 9, the valve assembly 120, according to an embodiment of the present disclosure, is illustrated. The valve assembly 120 is located in the instrument valve housing 110 and includes a centering mechanism 130, a guard assembly 140, the instrument seal 160, and a retainer 180. The centering mechanism 130 of the valve assembly 120 permits radial movement of the valve assembly 120 relative to a central longitudinal axis “X” of the instrument valve housing 110 in response to insertion of a surgical instrument (not shown) through the valve assembly 120 and radial movement of the surgical instrument relative to the central longitudinal axis “X”. In the absence of a surgical instrument or in the absence of radial movement of a surgical instrument relative to the central longitudinal axis “X”, the centering mechanism 130, as will be described in detail hereinbelow, returns the valve assembly 120 to a generally centered position such that a central opening 133 of the centering mechanism 130 and the central longitudinal axis “X” are coaxial. The guard assembly 140 protects the instrument seal 160 during insertion and withdrawal of a surgical instrument through the instrument seal 160, which, as discussed hereinabove, provides for sealed passage of the surgical instrument through the instrument valve housing 110. The retainer 180 includes first and second rings 182, 184 that are located on opposing sides of the centering mechanism 130 for maintaining relative positions of the guard assembly 140, the centering mechanism 130, and the instrument seal 160. Additionally, the retainer maintains 180 an aligned relationship of the guard assembly 140, the centering mechanism 130, and the instrument seal 160. In particular, the first ring 182 of the retainer 180 includes pins 186 that extend from a bottom surface of the first ring 182 while the second ring 184 of the retainer 180 includes complementary openings 188 for receiving the pins 186 of the first ring 182. The pins 186 may be releasably engaged with the openings 188 or the pins 186 may be secured within the openings 188 by welding, adhesives, friction fit, or other techniques as are known in the art. The pins 186 are insertable through bores 148 of the guard assembly 140, pores 138 of the centering mechanism 130, the holes 168 of the instrument seal 160, and the openings 188 of the second ring 184 of the retainer 180. This arrangement aligns the relative positions of the guard assembly 140, the centering mechanism 130, and the instrument seal 160. Although illustrated with pins 186 extending from the first ring 182 towards openings 188 in the second ring 184, the retainer may have the pins located on the second ring and the openings on the first ring. Alternatively, the first and second rings may have an alternating arrangement of pins and openings that are complementary such that the pins of one of the rings align with openings of the other of the rings allowing the rings to be attached to one another and define the retainer. The first ring 182 defines a central opening 185 extending therethrough and the second ring 184 defines a central opening 187 extending therethrough.

The centering mechanism 130 of the instrument valve housing 110 is configured to maintain the valve assembly 120 centered within the instrument valve housing 110. More particularly, the centering mechanism 130 includes an outer annular ring 132, an inner annular ring 134, and a bellows 136 disposed between the outer annular ring 132 and the inner annular ring 134. As shown in FIG. 2, the outer annular ring 132 is received between the inner housing section 116 and the lower housing section 114 to retain the centering mechanism 130 within the instrument valve housing 110. The inner annular ring 134 supports the guard assembly 140. For a detailed description of the structure and function of an exemplary centering mechanism, please refer to U.S. Pat. No. 6,702,787, the content of which is incorporated herein by reference in its entirety.

The guard assembly 140 may be formed from a sheet of a plastic or other suitable polymeric material by stamping with a tool that forms a ring 142 and blades 144a-d. The ring 142 surrounds the blades 144a-d and includes bores 148 extending therethrough for slidably receiving the pins 186 of the first ring 182 of the retainer 180. Further, when the valve assembly 120 is assembled, the guard assembly 140 is positioned between one side of the centering mechanism 130 and the first ring 182 of the retainer 180. The blades 144a-d are configured to flex towards the centering mechanism 130 in response to insertion of a surgical instrument (not shown) through a central orifice 146 of the guard assembly 140 and return to a generally planar configuration (i.e., parallel with the ring) once the surgical instrument is removed. The blades 144a-d extend towards a center of the ring 142 and define the central orifice 146 which has a diameter greater than an outside diameter of the surgical instrument.

During a surgical procedure utilizing cannula assembly 100, a surgical instrument (not shown) is introduced into the instrument valve housing 110 through the longitudinal passage 111 in the upper, lower, and inner housing sections 112, 114, 116. As described above, the distal end of the surgical instrument engages one or more of the blades 144a-d of the guard assembly 140 causing the blades 144a-d to flex downward into contact with the petals 162a-f of the instrument seal 160. This causes the central hole 176 of the instrument seal 160 to dilate such that the diameter of the central hole 176 is sufficiently large enough to accommodate passage of the surgical instrument therethrough. The guard assembly 140 minimizes damage to the instrument seal 160 during insertion and/or removal of the surgical instrument through the valve assembly 120. The guard assembly 140 operates to protect the instrument seal 160 and minimizes tearing or other damage as the surgical instrument is received through and withdrawn from the instrument seal 160.

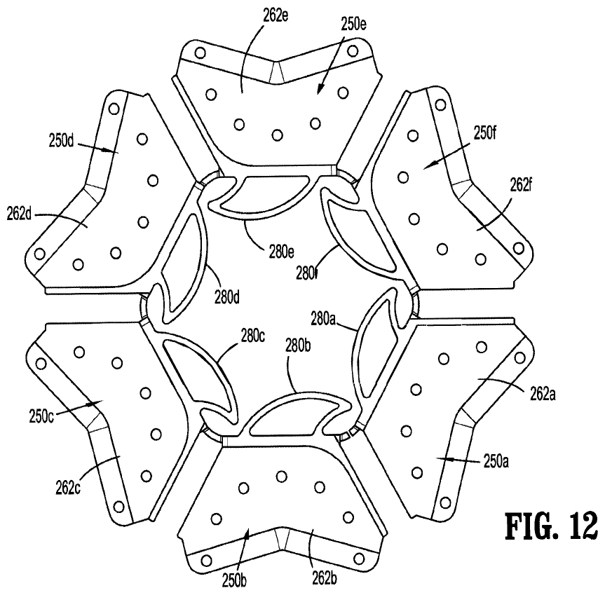

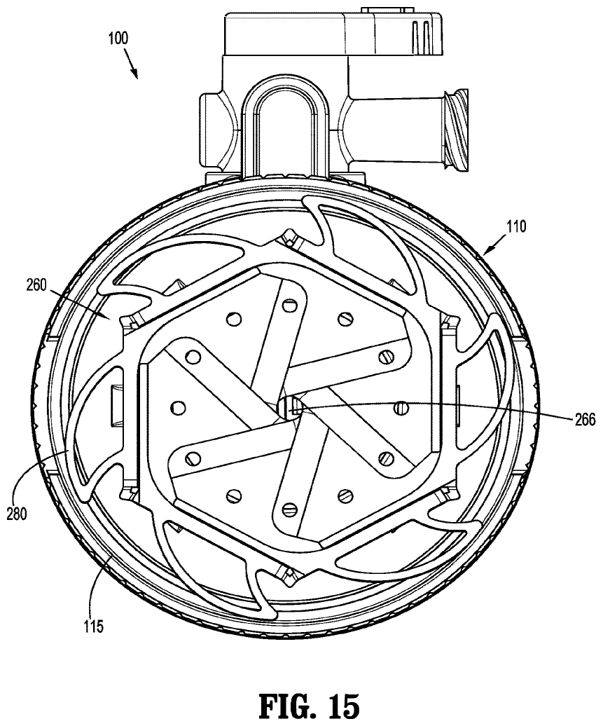

With reference now to FIGS. 10-12, an alternate embodiment of an instrument seal is illustrated and identified generally as instrument seal 260. Instrument seal 260 may be a direct replacement for both the instrument seal 160 and the centering mechanism 130 in valve assembly 120 as illustrated in the previous embodiment. The instrument seal 260, as illustrated, includes a frame 270 having six sides 272a-f. The frame 270 may have fewer sides (e.g., 4) or more sides (e.g., 8). Each side 272a-f is generally rectangular and extends along a length of a corresponding petal 262a-f. The number of petals 262 is equal to the number of sides 272 of the frame 270. Links 278 extend between adjacent side 272 defining a plurality of living hinges. In particular, links 278 define living hinges between sides 272a-b, between sides 272b-c, between sides 272c-d, between sides 272d-e, and between sides 272e-f. A gap is defined between sides 272a and 272f allowing sides 272a and 272f to move relative to each other. This arrangement facilitates folding the seal 270 thereby transitioning the seal 270 from the unfolded or initial configuration as shown in FIG. 12 to the folded or final configuration as shown in FIG. 10. Since sides 272a and 272f have a gap therebetween and lack a living hinge, one of sides 272a or 272f may be repositioned without disturbing the position of the other of sides 272a or 272f. The instrument seal 260 also includes a plurality of fins 280a-f that extends from respective sides 272a-f on the side of the side 272a-f opposite that of the petals 262a-f. Each fin 280a-f is a flexible and resilient structure that is normally biased towards a center of the unfolded instrument seal 260 (FIG. 12) and normally biased away from the center of the folded instrument seal 260 (FIG. 14). The biasing and resilience of the fins 280a-f acts to center the instrument seal 260 when the instrument seal is positioned in the valve housing 110 (FIG. 15) as will be discussed in further detail hereinbelow.

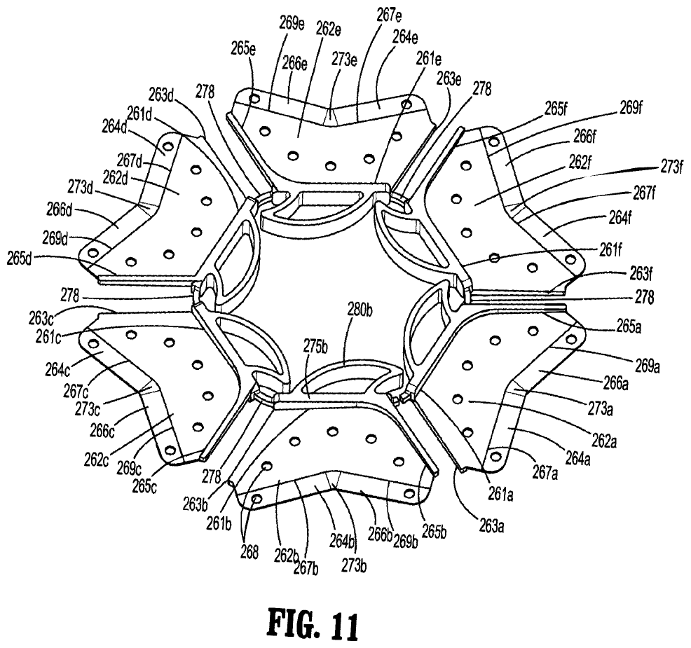

Each petal 262a-f is a five sided main panel 250a-f with holes 268 extending therethrough. Although shown with five sides, each main panel 250a-f may have more or less than five sides. A first or connection side 261a-f is coupled to a side or segment 272a-f of the frame 270. In the unfolded configuration (FIGS. 11 and 12), each petal 262a-f extends away from an outer surface of the frame 270 outside a perimeter defined by the frame 270. In the folded configuration (FIGS. 10 and 14), each petal 262a-f is bounded by the frame 270 and is within the perimeter defined by the frame 270. Each main panel 250a-f has angled second and third sides 263a-f, 265a-f that extend from the connection side 261a-f in a divergent manner. Fourth and fifth sides 267a-f, 269a-f of main panels 250a-f interconnect the angled second and third sides 263a-f, 265a-f. The fourth and fifth sides 267a-f, 269a-f of the main panels 250a-f of each petal 262a-f have equal lengths and are angled towards the corresponding connection side 261a-f such that they meet a point that would bisect the connection side 261a-f. Additionally, the fourth and fifth sides 267a-f, 269a-f are oriented such that they define an angle between 120° and 160°. First and second extenders 264a-f, 266a-f are attached to the fourth and fifth sides 267a-f, 269a-f. Each extender 264a-f, 266a-f includes a hole 268 extending therethrough. The extenders 264a-f, 266a-f and the main panels 250a-f of each petal 262a-f bend at a midpoint between the second and third sides 263a-f, 265a-f of each petal 262a-f such that, when viewed from the end (i.e., from the extenders towards the connection side) (similar to FIG. 5), the petal 62a-f has a slight curvature of about 5° to about 10°.

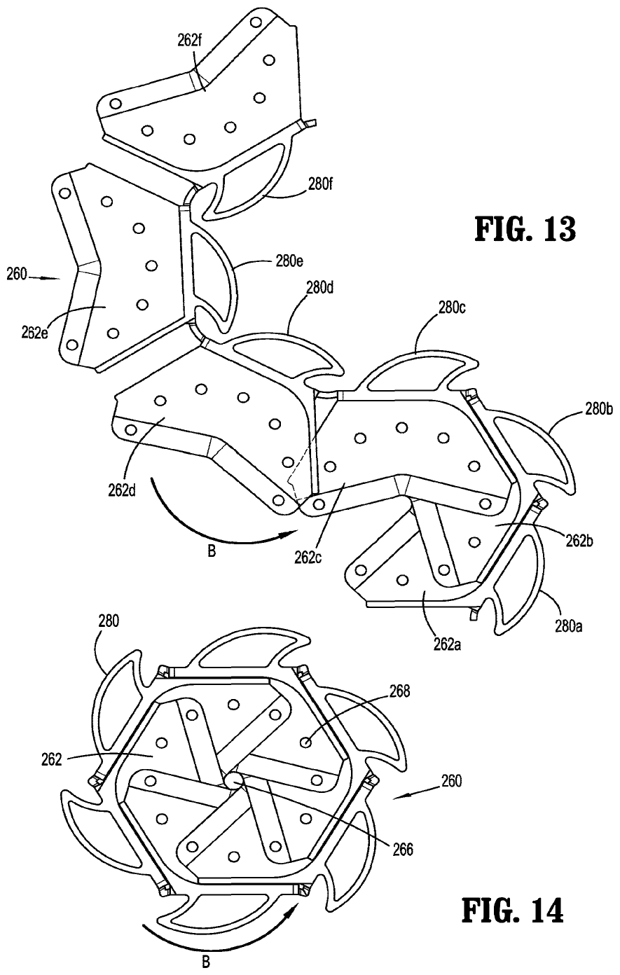

The first petal 262a is folded by pivoting the first side 272a and the first petal 262a about the living hinge defined by the link 278 that is disposed between the first and second sides 272a, 272b in the direction of arrow “B”. As such, the first petal 262a partially overlaps the second petal 262b. Subsequently, the first and second petals 262a, 262b are pivoted by pivoting the second side 272b about the living hinge defined by the link 278 that is disposed between the second side 272b and the third side 272c such that the second petal 262b partially overlaps the third petal 262c (FIG. 13). Next, the first, second, and third petals 262a-c are pivoted by pivoting the third side 272c about the living hinge defined by the link 278 that is disposed between the third side 272c and the fourth side 272d such that the third petal 262c partially overlaps the fourth petal 262d. Subsequently, the first, second, third, and fourth petals 262a-d are pivoted by pivoting the fourth side 272d about the living hinge defined by the link 278 that is disposed between the fourth side 272d and the fifth side 272e such that the fourth petal 262d partially overlaps the fifth petal 262c. The first, second, third, fourth, and fifth petals 262a-e are pivoted by pivoting the fifth side 272e about the living hinge defined by the link 278 that is disposed between the fifth side 272e and the sixth side 272f such that the fifth petal 262e partially overlaps the sixth petal 262f and the sixth petal 262f partially overlaps the first petal 262a. The fully folded seal 260 is illustrated in FIG. 14. All the folds occur in the direction identified by arrow “B”.

After all the petals 262a-f are folded, a center orifice 266 is defined and is configured to engage an outer surface of a surgical instrument (not shown) inserted through the seal 260 such that the center orifice 266 surrounds the surgical instrument in a sealing manner to inhibit the passage of insufflation fluids and defines a fluid tight barrier. Further, once the petals 262a-f are folded over, the holes 268 of the petals 262a-f are aligned thereby allowing pins 186 of the retainer 180 to pass through the holes 268. In this embodiment, the pins 186 are insertable through bores 148 of the guard assembly 140, the holes 268 of the instrument seal, and the openings 188 of the second ring 184 of the retainer 180. This arrangement aligns the relative positions of the guard assembly 140 and the instrument seal 260. Although illustrated with pins 186 extending from the first ring 182 towards openings 188 in the second ring 184, the retainer may have the pins located on the second ring and the openings on the first ring. Alternatively, the first and second rings may have an alternating arrangement of pins and openings that are complementary such that the pins of one of the rings align with openings of the other of the rings allowing the rings to be attached to one another and define the retainer.

As each petal 262a-f at least partially overlaps a first adjacent petal 262 and is at least partially overlapped by a second adjacent petal 262, the petals 262a-f of the seal are interwoven. This interwoven arrangement of the petals 262a-f facilitates the seal 260 maintaining its shape during insertion and withdrawal of a surgical instrument through the center orifice 266. For example, with additional reference to FIG. 2, the seal 260 would replace the seal 160 and the centering mechanism 130. FIG. 15 illustrates the placement of the instrument seal 260 in vale housing 110 of the cannula assembly 100. During insertion of the surgical instrument through the valve housing 110 of the surgical access assembly 100, a shaft of the surgical instrument passes through the central opening 185 of the first ring 182, the central orifice 146 of the guard assembly 140, the center orifice 266 of the instrument seal 260, and the central opening 187 of the second ring 184. As the shaft of the surgical instrument passes through the center orifice 266 of the seal 260 during insertion, the petals 262a-f of the seal 260 flex towards the second ring 184 and surround an outer surface of the shaft of the surgical instrument providing a fluid tight barrier between the petals 262a-f of the seal 260 and the shaft of the surgical instrument. During withdrawal of the surgical instrument, the petals 262a-f of the seal 260 flex towards a proximal portion of the valve housing 110 in response to proximal movement of the shaft of the surgical instrument. The petals 262a-f of the seal 260 resiliently return to their initial or rest configuration (FIG. 10) once the shaft of the surgical instrument is removed from the center orifice 266 of the seal 260. Due to the petals 262a-f being interwoven, they return to their initial configuration. In the event that the petals 262a-f have slightly different rates of movement, the interwoven arrangement of the petals 262a-f results in the slowest moving petal 262 acting as a governor and limiting the rate of movement of the remaining petals 262. This tends to maintain contact between the petals 262a-f and the outer surface of the shaft of the surgical instrument thereby maintaining the fluid tight boundary of the seal 260 with respect to the surgical instrument during movement of the shaft relative to the seal 260.

Referring now to FIG. 15, the instrument seal 260 is positioned in the valve housing 110 and the fins 280a-f contact an inner wall 115 of the valve housing 110. In an initial state, the normal biasing force exerted by the fins 280a-f act to center the instrument seal 260 in the valve housing 110 such that the center orifice 266 is aligned with the central longitudinal axis “X” of the cannula assembly 100 (FIG. 2). When a surgical instrument is inserted through the valve housing 110 and the center orifice 266, any radial movement of the surgical instrument relative to the longitudinal axis “X” moves the instrument seal 260 in the same radial direction. This results in the center orifice 266 being radially offset from the central longitudinal axis “X”. In particular, when the instrument seal 260 is moved radially, the fins 280a-f in the direction of movement are compressed more while the fins 280a-f on the opposing side a relaxed more. Thus, when the force is removed, the compressed fins 280a-f will move towards their initial position and return the instrument seal 260 to its at rest position where the center orifice 266 is aligned with the central longitudinal axis “X”. It is contemplated that all of the fins 280a-f will be slightly compressed when the instrument seal 260 is disposed within the valve housing 110.

Each petal 262a-f is connected to a corresponding side 272a-f of the frame 270 along a first or connection side 261a-f. Each petal 262a-f also includes angled second and third sides 263a-f, 265a-f that extend from the corresponding connection side 261a-f in a divergent manner. Fourth and fifth sides 267a-f, 269a-f of each petal 262a-f interconnect the angled second and third sides 263a-f, 265a-f. The fourth and fifth sides 267a-f, 269a-f of the petals 262a-f have equal lengths and are angled towards the corresponding connection side 261a-f such that they meet at a point that would bisect the connection side 261a-f. The fourth and fifth sides are oriented such that they that they define an angle of 150°. The fourth and fifth sides may define an angle between about 120° and about 165°. First and second extenders 262a-f, 264a-f are attached to the fourth and fifth sides 267a-f, 269a-f. The first and second extenders 262a-f, 264a-f have equal lengths and meet at a taper 273a-f that also is located at a point that would bisect the corresponding connection side 261a-f.

It will be understood that various modifications may be made to the embodiments disclosed herein. Therefore, the above description should not be construed as limiting, but merely as exemplifications of particular embodiments. Those skilled in the art will envision other modifications within the scope and spirit of the claims appended hereto.

1. (canceled)

2. A surgical access assembly comprising:
a housing comprising a central axis through a longitudinal passage;
a valve assembly movable radially within the longitudinal passage of the housing, the valve assembly comprising an instrument seal and a guard;
a centering ring within the housing and having an outer annular ring that surrounds the valve assembly; and
a retainer comprising a plurality of pins passing through corresponding bores in the guard, the centering ring, and the instrument seal,
wherein the centering ring permits radial movement of the valve assembly relative to the central axis of the housing.

3. The surgical access assembly of claim 2, wherein the centering ring comprises a bellows.

4. The surgical access assembly of claim 3, wherein the bellows returns the valve assembly to a centered position in which a central opening of the centering ring and the central axis of the housing are coaxial.

5. The surgical access assembly of claim 3, wherein the centering ring comprises an inner annular ring that supports the guard.

6. The surgical access assembly of claim 2, wherein the instrument seal comprises a frame connected to a plurality of petals.

7. The surgical access assembly of claim 6, wherein the frame is integrally formed as a monolithic structure.

8. The surgical access assembly of claim 6, wherein the frame forms a boundary that defines a passage through the instrument seal.

9. The surgical access assembly of claim 6, wherein the plurality of petals fold in an interlocking pattern into a folded configuration.

10. The surgical access assembly of claim 6, wherein the plurality of petals flex in a distal direction during insertion of a surgical instrument through the longitudinal passage.

11. The surgical access assembly of claim 2, wherein the retainer maintains an aligned relationship of the guard, the centering ring, and the instrument seal.

12. The surgical access assembly of claim 11, wherein the retainer comprises a first ring having the plurality of pins, and a second ring having a plurality of openings that aligns with the plurality of pins.

13. The surgical access assembly of claim 2, wherein the corresponding bores in the instrument seal, the guard, and the centering ring comprise a set of bores in the guard, a set of pores in the centering ring, and a set of holes in the instrument seal.

14. A surgical access assembly comprising:
a housing comprising a central axis through a longitudinal passage;
a cannula having a proximal end attached to the housing and an opposite distal end;
a valve assembly movable radially within the longitudinal passage of the housing, the valve assembly comprising an instrument seal and a guard, wherein the instrument seal comprises four or more petals foldable around a central hole;
a centering ring having an outer annular ring that surrounds the valve assembly; and
a retainer comprising a first ring and a second ring, the first ring having a plurality of pins passing through corresponding openings in the second ring,
wherein the instrument seal, the guard, and the centering ring are sandwiched between the first and second rings, and
wherein the centering ring permits radial movement of the valve assembly relative to the central axis of the housing.

15. The surgical access assembly of claim 14, wherein the four or more petals are movable between unfolded, partially folded, and folded configurations.

16. The surgical access assembly of claim 14, wherein the plurality of pins pass through corresponding bores in the guard, the centering ring, and the instrument seal.

17. The surgical access assembly of claim 14, wherein the plurality of pins align relative positions of the guard, the centering ring, and the instrument seal.

18. A surgical access assembly comprising:
a housing comprising a central axis through a longitudinal passage;
a valve assembly movable radially within the longitudinal passage of the housing, the valve assembly comprising an instrument seal and a guard, wherein the instrument seal comprises four or more petals foldable about a seal frame around a central hole;
a plurality of flexible fins connected to the seal frame and contacting an inner wall of the housing; and
a retainer comprising a first ring and a second ring, the first ring having a plurality of pins passing through corresponding openings in the guard, the instrument seal, and the second ring, wherein the instrument seal and the guard are sandwiched between the first and second rings.
